(19) United States
(12) Patent Application Publication
(10) Pub. No.: US 2017/0035333 A1
(43) Pub. Date: Feb. 9, 2017

(54) Apparatus for Measuring a Vital Function of a Living Creature

(71) Applicant: OSRAM Opto Semiconductors GmbH, Regensburg (DE)

(72) Inventor: Rolf WEBER, San Jose, CA (US)

(73) Assignee: OSRAM Opto Semiconductors GmbH, Regensburg (DE)

(21) Appl. No.: 14/817,919

(22) Filed: Aug. 4, 2015

(51) Int. Cl. A61B 5/1455

(52) U.S. Cl. CPC A61B 5/14552; A61B 2562/0238

(57) ABSTRACT

Apparatus for measuring a vital function of a living creature comprising: a light source, wherein the light source emits a first light signal and a second light signal; a receiver, wherein the receiver receives a reflected first light signal, wherein the receiver receives a reflected second light signal; an evaluating unit, to evaluate the received first light signal with the received second light signal, wherein the evaluating unit measures the vital function based on the evaluated received first light signal.

### FIELD OF THE INVENTION

The proposed apparatus is able to measure a vital function of a living creature with a light source, wherein the light source is embodied to emit a first light signal and a second light signal. The two reflected light signals were used to measure the vital function.

### BACKGROUND OF THE INVENTION

It is known in the state of the art to measure a heart rate by using a light source that sends a light signal to a human being and the reflected light signal is evaluated for measuring the heartbeat.

### SUMMARY OF THE INVENTION

One aspect of the invention is directed to an apparatus for measuring a vital function of a living creature comprising:

a light source, wherein the light source is embodied to emit a first light signal and a second light signal; a receiver, wherein the receiver is embodied to receive a reflected first light signal, wherein the receiver is embodied to receive a reflected second light source; an evaluating unit, wherein the evaluating unit is embodied to evaluate the received first light signal with the received second light signal, wherein the evaluating unit is embodied to measure the vital function based on the evaluated received first light signal.

Another aspect of the invention is directed to a method for measuring a vital function of a living creature with the following steps: a first light signal and a second light signal are sent to the living creature; a reflected first light signal and a reflected second light are received; the received first light signal is evaluated with the received second light signal; the evaluated first light signal represents the vital function.

The proposed apparatus provides the advantage that a more precise measuring of a vital function is possible. This advantage is attained by providing a first light signal for measuring the vital function and a second light signal that is used for evaluating the received first light signal. The considering of the received second light signal improves the evaluation of the evaluated received first light signal. The evaluation of the received first light signal by the received second light signal provides for example the advantage that a change of distance between the apparatus and the living creature can be considered. Therefore it is possible to reduce the effect of the change of distance between the apparatus and the living creature for the measuring of the vital function.

Furthermore, the light source is embodied by two different light sources, wherein the first light source provides the first light signal, and wherein the second light source provides the second light signal. The two different light sources for the two light signals provide the advantage that the two light signals can be controlled independently. Furthermore, two specific first and second light signals can be provided. Additionally, the two different light sources can be controlled independently.

Therefore, the two different light sources may emit the corresponding light signal with different light intensity. Furthermore, the two different light sources may provide two different light signals with different wavelengths and/or with different wavelength spectra. Therefore, a higher flexibility is attained by using two different light sources.

The first and/or the second light source may be embodied as light emitting diodes. The first and/or the second light source may be embodied as laser diodes.

The first light source may emit a light signal with a wavelength between 497 nm and 530 nm which means green light.

The second light source may emit a light signal with a wavelength between 450 nm and 482 nm which means blue light.

According to one aspect the first light signal may be more sensitive than the second light signal with regard to the vital function that is measured. For example the first light signal may show a higher reflectivity for the measured vital function. For example if a heartbeat of a living creature for example a human being is measured, then the first light signal should be more sensitive referring to a reflection by blood than the second light signal.

Depending on the vital function that is measured different first and/or second light signals may be used. For example a blood oxygen level or a heartbeat is measured.

According to a further aspect the second light signal may comprise a blue color light or may mainly comprise light with blue color. Blue color light is less sensitive referring to a reflection by blood. The first light signal may comprise a green color light or may mainly comprise a green color light. Green color light is more sensitive to a reflection by blood. Therefore a change of blood volume can be more easily detected by a green color first light signal. This means that the reflected second light signal changes less depending on the blood volume compared to the first light signal. The second and the first light signal may have a similar or identical sensitivity with regard to a change of distance between the apparatus and the body of the living creature.

The evaluating unit may be embodied to normalize an amplitude of the received first light signal by an amplitude of the received second light signal. The normalization of the amplitude of the received first light signal has the advantage that the normalized received first light signal is less dependent on other influences, for example a change of distance between the apparatus and the body of the living creature or other effects that do not depend on the vital function. Therefore, it is possible to identify more clearly a change of the received first light signal for example a change of the amplitude of the received first light signal that is caused by the measured vital function.

The evaluating unit may use other techniques to increase the quality of the received first light signal by using the received second light signal in contrast to normalization. There may be further evaluating methods that assist the evaluation of the first received light signal by using the received second light signal.

The evaluating unit may be embodied to determine as a vital function a heartbeat rate. This means that a change of blood volume is detected by the received first light signal.

The evaluating unit may be embodied to determine as a vital function a blood oxygen concentration by evaluating the received first light signal.

The first light source and the second light source may be arranged abreast which means side by side. Therefore the first light signal and the second light signal travel nearly on the same way from the light sources to the living creature and back to the receiver. Therefore the first light signal and the second light signal are sent with nearly the same distance from the apparatus to the body of the living creature and back to the receiver. Therefore an influence of change of distance between the apparatus and the body is exactly or at least nearly the same for the first light signal and the second light signal. Therefore a more precise evaluation of the received first light signal by the received second light signal is possible compared to an arrangement with distant first and second light source.

The first and the second light signal may be embodied in that way that the first light signal is reflected with a higher degree by blood than the second light signal.

The reflected light intensity of the first light signal and the reflected light intensity of the second light signal may change identical or similar with a change of the distance between the light source and the body of the living creature.

### BRIEF DESCRIPTION OF THE DRAWINGS

The above-described properties, features and advantages of the present invention as well as the manner in which these are achieved will be explained more clearly and distinctly in connection with the following description of the embodiment examples which will be explained in more detail in conjunction with the drawings. Thereby, in a highly schematic illustration,

### DETAILED DESCRIPTION OF THE DRAWINGS

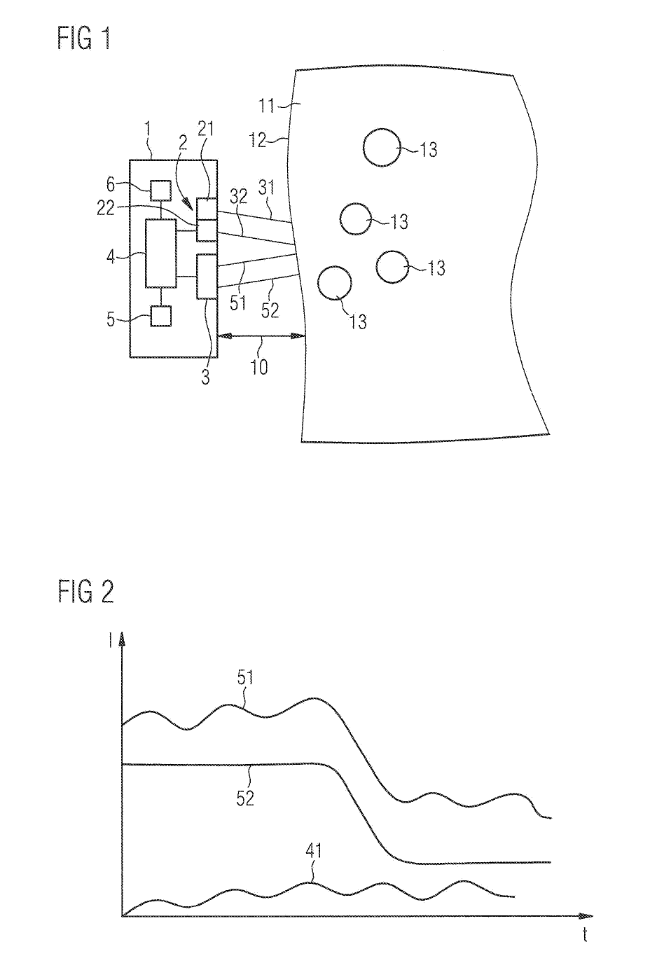

FIG. 1 depicts an arrangement with an apparatus and a wrist of a human being, and

FIG. 2 depicts a diagram for a received first light signal, a received second light signal and an evaluated received first light signal.

FIG. 1 depicts in a schematic view an apparatus 1 that comprises a light source 2, a receiver 3 and an evaluating unit 4. The evaluating unit 4 is connected with the light source 2 and the receiver 3. Additionally there may be a display 5 and/or a memory 6 and/or an interface 7. The evaluating unit 4 is connected with the display 5 and/or the memory 6 and/or the interface 7. Depending on the used embodiment, the light source 2 may be embodied as a first light source 21 and a second light source 22. At a distance 10 to the apparatus 1 a body of living creature, for example a body of a human being is arranged. For example a wrist 12 of a human being may be arranged at a given distance 10 to the apparatus 1. In the wrist 12 there are blood vessels 13. The apparatus 1 may be part of a belt or connected with a belt or may be part or connected with a wrist band. The evaluating unit 4 may control the light source 2 or the first and second light source 21, 22 with regard to an operation parameter of the light source 2 or the first and second light source 21, 22. The operating parameter may be a wavelength, a wavelength spectrum and/or a light intensity.

The light source 2 emits a first light signal 31 and a second light signal 32. In the embodiment with the first and the second light source 21, 22 the first light source 21 emits the first light signal 31 and the second light source 22 emits the second light signal 32.

The two light sources 31, 32 may emit the light signal sources 31, 32 with different light intensities. Furthermore the two different light sources may provide two light signals with two different wavelengths and/or with two not identical wavelength spectra. The first and/or the second light source may be embodied as light emitting diodes. The first and/or the second light source may be embodied as laser diodes.

The first light source may for example emit a light signal with a wavelength between 497 nm and 530 nm which means green light. The second light source may for example emit a light signal with a wavelength between 450 nm and 482 nm which means blue light.

The first light signal may be more sensitive than the second light signal with regard to the vital function that is measured. The first light signal may show a higher reflectivity for the measured vital function. For example if a heartbeat of a living creature for example a human being is measured, then the first light signal should be more sensitive referring to a reflection by blood than the second light signal.

The first and the second light signals 31, 32 are sent to the wrist 12. The first and the second light signal 31, 32 are reflected by the wrist 12 especially by the blood vessels 13. The reflected first and second light signal 51, 52 are received by the receiver 3. In a simple embodiment, the receiver 3 may detect amplitudes of the received first and second light signals 51, 52. In a further embodiment the receiver 3 may detects light spectra of the received first and second light signals 51, 52. The received first and second light signals 51, 52 are forwarded by the receiver 3 to the evaluating unit 4.

The reflected light intensity of the first light signal and the reflected light intensity of the second light signal may change identical or similar with a change of the distance between the light source and the body of the living creature.

The evaluating unit 4 uses the received second light signal 52 for evaluating the received first light signal 51. There may be different methods to evaluate the received first light signal 51 by the received second light signal 52. For example a normalization of the received first light signal 51 by the received second light signal 52 may be used as an evaluation. A basic idea is to reduce the influence of parameters that do not directly correspond to the vital function that should be measured. For example a change of distance 10 between the apparatus 1 and the body of the living creature for example a wrist 12 of a human being influences the received first light signal 51 that is measured by the receiver 3.

The second light signal may comprise a blue color light or may mainly comprise light with blue color. Blue color light is less affected by the blood volume than for example green color light. The first light signal may comprise a green color light or may mainly comprise a green color light. Green color light has a higher reflectance by blood. Therefore a change of blood pressure and blood volume can be more easily detected by a green color first light signal. This means that the reflected blue second light signal changes less depending on the blood volume compared to the green first light signal. The second and the first light signal may have a reflectance with a similar or an identical dependence with regard to a change of distance between the apparatus and the body of the living creature.

As a vital function for example a heart rate may be measured. The heart rate causes a change of a volume of blood in the blood vessels 13. The change of blood volume results in a change of reflected light intensity of the first light signal. The second light signal may be chosen in that way that a change of blood volume in the blood vessels 13 causes less change in the reflected light intensity compared to the first light signal. Furthermore a change of distance 10 between the apparatus 1 and the wrist 12 may cause a change of reflected first light signal and reflected second light signal in nearly the same way. Furthermore, if another vital function should be measured for example a concentration of oxygen in the blood, then also the second light signal should be chosen in such a way that a change of oxygen concentration the blood has less influence on the reflected light intensity of the second light signal compared to the first light signal.

The evaluating unit 4 uses the received second light signal 32 to eliminate at least partially an influence of change of the distance 10 between the apparatus and the wrist on the received reflected first light signal 51. This may for example be done by subtracting the normalized received second light signal from the received first light signal. For example a light intensity of the received second light signal is subtracted from the light intensity of the received first light signal.

The first light signal comprises a dc portion that may basically depend on a distance between the sensor and the body. The first light signal additionally comprises an ac portion carrying a change of the vital function for example a heart rate information. In case of an air gap change between the sensor and the skin of the living creature, the dc portion of the first light signal and the reference second light signal show the same relative change in signal strength caused by the change of air gap. So when the second light signal changes by 50% one can expect that the dc portion of the first light signal also changes an amount close to 50% as well. This means if the signal strength of the second light signal falls by 50%, then one can subtract 50% from the signal strength of the first light signal. So it is easier to detect the change of the strength of the first light signal that depends on the change of the vital function of the living creature.

The evaluated first light signal 41 more clearly shows the influence of the change of blood pressure or the change of oxygen concentration of the blood. Thus it is possible for the evaluating unit 4 to measure more precisely the heartbeat rate and/or the oxygen concentration.

In a further embodiment, the receiver 3 may have a wavelength filter to select the first and the second reflected light signal 51, 52 and to reduce the effect of ambient light. The receiver 3 may receive the first and second light signals by using two independent receivers with two different wavelength filter. Furthermore there may be only one receiver the senses the first and the second light signals one after the other by using the two different wavelength filter. Therefore it is possible to prevent or at least decrease a cross talk between the received first light signal and the received second light signal 51, 52 if the lights are not operated in sequence.

The evaluating unit 4 may show the measured vital function on the display 5 and/or store the measured vital function in the memory 6 and/or send the measured vital function by the interface 7 to another apparatus.

The interface 7 may also be used to read out the stored vital function of the memory 6. Depending on the used embodiment, the received first and second light signal 51, 52 that are forwarded by the receiver 3 to the evaluating unit 4 are also stored in the memory 6. Therefore an evaluation of the received first light signal 51 considering the received second light signal 52 may also be performed by another evaluating unit in another apparatus. The stored data may be transmitted by the interface 7 to the other evaluating unit.

FIG. 2 depicts in a schematic view a diagram with a received first light signal 51, a received second light signal 52 and an evaluated first light signal 41. The diagram shows the light intensity I of the received first and second light signal 51, 52 and the light intensity I of the evaluated first light signal 41 over the time t. The evaluated first light signal 41 is for example the difference between the received first light signal 51 and the received second light signal 52. The distance between the body and the apparatus and the body of a living creature is increased. As a result, the intensity I of the reflected first and second light signal 51, 52 decreases. However a mean value of the intensity of the evaluated first signal 41 does nearly not change.

Since the influence of a change of blood volume or oxygen level to a change of reflection of the first light signal is low and may be in an area of 1% or 2% of the reflectance. Therefore the evaluation increases the quality of measurement. For example a small change of 5% of the distance 10 between the apparatus 1 and the wrist 12 may cause a change of 30% of the intensity of the reflected first light signal 51 that is received by the receiver 3. Therefore it may be helpful to consider a change of distance with the received second light signal 52.

The invention has been illustrated and described in detail on the basis of the preferred embodiment examples. However, the present invention is not limited to the disclosed examples. Rather, other variations may be derived therefrom by a person skilled in the art without exceeding the protective scope of the invention.

1. Apparatus for measuring a vital function of a living creature comprising:
a light source, wherein the light source is configured to emit a first light signal and a second light signal;
a receiver, wherein the receiver is configured to receive a reflected first light signal, wherein the receiver is configured to receive a reflected second light signal; and
an evaluating unit, wherein the evaluating unit is configured to evaluate the received first light signal with the received second light signal, wherein the evaluating unit is configured to measure the vital function based on the evaluated received first light signal.

2. The apparatus of claim 1, wherein the light source comprises two light sources, wherein the first light source provides the first light signal, and wherein the second light source provides the second light signal.

3. The apparatus of claim 1, wherein the light source and/or the first light source and/or the second light source are light emitting diodes or laser diodes.

4. The apparatus of claim 1, wherein the first light signal comprises light with green color, and/or wherein the second light signal comprises light with blue color.

5. The apparatus of claim 1, wherein the first light signal is light with green color, and/or wherein the second light signal is light with blue color.

6. The apparatus according to claim 1, wherein the evaluating unit is configured to normalize an amplitude of the received first light signal by an amplitude of the received second light signal.

7. The apparatus of claim 1, wherein the evaluating unit is configured to determine as vital function a heart beat rate.

8. The apparatus of claim 1, wherein the evaluating unit determines as a vital function a blood oxygen concentration.

9. The apparatus of claim 2, wherein the first light source and the second light source are arranged side by side.

10. The apparatus of claim 1, wherein the first and the second light signal are configured so that the first light signal is reflected with a higher degree by blood than the second light signal.

11. The apparatus of claim 1, wherein a reflected light intensity of the first light signal and a reflected light intensity of the second light signal may change identical or similar with a change of the distance between the light source and the body of the living creature.

12. Method for measuring a vital function of a living creature comprising:
sending a first light signal and a second light signal to the living creature;
receiving a reflected first light signal and a reflected second light signal; and
evaluating the received first light signal with the received second light signal;
wherein the evaluated first light signal represents the vital function.
